SEQUENCE LISTING (2 sequences)

SEQ ID NO: 1 - protein, 212 aa, Carica papaya
IPEYVDWRQKGAVTPVKNQGSCGSCWAFSAVVTIEGIIKIRTGNLNEYSEQELLDCDRRSYGCNGGYPWSALQLVAQYGIHYRNTYPYEGVQRYCRSREKGPYAAKTDGVRQVQPYNEGALLYSIANQPVSVVLEAAGKDFQLYRGGIFVGPCGNKVDHAVAAVGYGPNYILIKNSWGTGWGENGYIRIKRGTGNSYGVCGLYTSSFYPVKN

SEQ ID NO: 2 - protein, 213 aa, Ananas comosus
AVPQSIDWRDYGAVTSVKNQNPCGACWAFAAIATVESIYKIKKGILEPLSEQQVLDCAKGYGCKGGWEFRAFEFIISNKGVASGAIYPYKAAKGTCKTDGVPNSAYITGYARVPRNNESSMMYAVSKQPITVAVADANANFQYYKSGVFNGPCGTSLNHAVTAIGYGQDSIIYPKKWGAKWGEAGYIRMARDVSSSSGICGIAIDPLYPTLEE

(19) United States
(12) Patent Application Publication
(10) Pub. No.: US 2007/0275045 A1
(43) Pub. Date: Nov. 29, 2007

(54) Composition for the treatment of warts and molluscum contagiosum

(76) Inventors: Robin D. Evans, Stamford, CT (US); Joel Evans, Stamford, CT (US)

Correspondence Address: EDWARDS ANGELL PALMER & DODGE LLP, P.O. BOX 55874, BOSTON, MA, 02205, US

(21) Appl. No.: 11/712,064

(22) Filed: Feb. 28, 2007

Related U.S. Application Data
(60) Provisional application No. 60/777,667, filed on Feb. 28, 2006.

(51) Int. Cl. A61K 9/70; A61K 38/46; A61P 17/12

(52) U.S. Cl. 424/449; 424/94.65

(57) ABSTRACT

The present invention provides compositions comprising mixtures of papain and bromelain, combined with a pharmaceutically acceptable carrier, for treating viral-induced skin lesions. The present invention further provides methods of treating viral-induced skin lesions by administering to a subject in need thereof a therapeutically effective amount of papain and bromelain.

### CROSS-REFERENCE TO RELATED APPLICATIONS

This application is entitled to the benefit under 35 U.S.C. 119(e) and 37 C.F.R. 1.78(a)(4)-(a)(6) of the filing date of U.S. provisional application Ser. No. 60/777,667, filed Feb. 28, 2006.

Any and all references cited in the text of this patent application, including any U.S. or foreign patents or published patent applications, International patent applications, as well as, any non-patent literature reference, including any manufacturer's instructions, are hereby expressly incorporated by reference.

### BACKGROUND OF THE INVENTION

1. Field of the Invention

The present invention relates to compositions and methods for treating lesions caused by viral infections of the epidermis. The compositions and methods of the invention, in particular, are effective against the treatment of benign epidermal lesions, such as, for example, warts and/or molluscum contagiosum. The compositions of the invention relate to mixtures of bromelain and papain, compounded with a pharmaceutically acceptable carrier for direct application to the lesions of interest, for example, by topical application, transdermal application, or direct injection.

2. Background of the Related Art

Viral infections of the epidermis can cause an array of skin disorders, the exact nature of which depends in large part on the particular infectious agent, the area on the body of the infected skin, and the age and health of the infected subject. Two widespread viral-induced skin conditions include warts and molluscum contagiosum.

Warts are a common affliction, estimated to affect as many as seven to twelve percent of the population. Human papillomavirus (HPV) is the causative agent of warts. Once developed, warts can be spread to other parts of the body or to other persons through skin-to-skin contact or contact with a surface contaminated with HPV. Most warts generally do not lead to serious disease, but some warts, including especially genital warts, have been associated with malignant cancers. Common warts affecting nongenital areas are generally not thought to have malignant potential. While there are over 60 serotypes of HPV, determining the particular serotype involved in the infection is generally sought only for genital infections as knowledge of the serotype can facilitate treatment and care. Warts are more commonly diagnosed based on their physical appearances and locations on the body.

Generally, five different types of warts are classified by their clinical presentation. Common warts (i.e. verrucae vulgaris) are domed, irregularly surfaced lesions that display hyperkeratosis and may occur anywhere on the body, but typically not including genital regions, mucous membranes or plantar surfaces (soles or bottoms of the feet). Periungual warts occur on the skin surrounding finger and toe nails. Permanent nail deformity can occur where the wart physically contacts the nail or invades the nail space from the underside. Deformity can also occur when the wart involves the nail folds or cuticles or affects the nail matrix. Flat warts (i.e. verrucae planae) usually occur on the face, trunk and extremities. They often occur on the faces and extremities of children and on the lower legs of women. Plantar warts (i.e. verrucae plantares) occur only on the soles of the feet. Such warts typically become callused and grow into the foot due to the forces exerted on the foot from everyday movement. Plantar warts may often be associated with pain. Condylomata acuminata are venereal warts that occur on the genitals, including the mucous membranes. Premalignant warts (i.e. epidermodysplasia verruciformis) usually occur on the hands and feet and are rare in occurrence.

Warts can cause pain and discomfort, and may lead to complications, including, in some cases, cancer, if left untreated or treated improperly. Methods for destroying or treating warts are varied, and include mechanical destruction methods, chemical therapies, and immunotherapies. For example, warts can be removed mechanically by surgical excision, tissue freezing methods (e.g. liquid nitrogen freeze via spray gun or cotton-tipped application at −196° C. or cryogen with nitrous oxide tank freeze at −89° C.), burning methods (e.g. laser treatment, electrocautery, electrodesiccation), and cutting methods (e.g. curettage or surgical excision). Warts can be treated chemically, for example, by nonprescription salicylic acid (e.g. COMPOUND W® or DUOFILM™ liquid or patches), bichloroacetic acid, trichloroacetic acid, tretinoin (Retin-A), dinitrochlorobenzene, cantharidin, podophyllin, 5-fluorouracil and bleomycin by way of topical or intralesional injections. Such treatments, especially acid treatments, require daily application for extended periods of time, ranging from weeks to many months, and further requires a continual removal of dead skin tissue. Newer treatment of warts involves methods of enhancing a patient's immune system, for example, by administering the topical agent imiquimod (Aldara), interferons (e.g. interferon alpha-2b or interferon alpha-N3), antigens (e.g. Candida antigen or mumps antigen), or cimetidine. Typically, immunotherapies are sought to suppress HPV re-infection once the gross lesions have been destroyed.

These known treatments have a variety of disadvantages. Such disadvantages include ineffectiveness (e.g. chemical treatments and cryotherapy), undesirable pain and prolonged recovery (e.g. cryotherapy and burning methods), undesirable scarring (e.g. surgical excision), and high expense (e.g. bleomycin injections to cause acute tissue necrosis). Pain is especially undesirable and difficult to manage in children. Another disadvantage, especially with child patients, is that many of the current treatments require multiple visits and/or applications making it difficult and impractical for families to carryout therapies. In addition, many of the standard therapies are unsuccessful at preventing lesions from reoccurring. Significantly, the standard treatments have not been found to be uniformly effective in treating warts and preventing recurrences.

As with warts, molluscum contagiosum is a skin condition resulting from epidermal skin viral infections and have characteristic epidermal lesions. The causative agent is generally regarded as the molluscum contagiosum virus (MCV), which is a DNA virus in the Poxviridae family. Among sexually active adults, molluscum contagiosum is considered a sexually transmitted disease and is generally transmitted by skin-to-skin contact. Molluscum contagiosum is also a commonly occurring condition in children and in patients who are immunocompromised, e.g. HIV-infected persons.

Molluscum contagiosum can be diagnosed based on the characteristics of the lesions. The typical molluscum contagiosum lesion is an asymptomatic, firm, smooth, round papule with central umbilication. The lesions are generally 3 to 5 mm in diameter and occur in batches less than about 30. In children, molluscum contagiosum is most prevalent on the face, trunk and the extremities. In adults, the lesions are most commonly found in the lower abdomen and in the genital regions, and they are spread through sexual contact. It is generally a self-limited disease in immunocompetent persons, but may persist for many weeks or months or even years if left untreated. In immunocompromised persons, the condition is generally not self-limiting and infection can be much more severe.

Left untreated, molluscum contagiosum lesions can disappear with no residual scarring. One common method for addressing molluscum contagiosum is by local destruction of the lesions, in part to prevent spreading to other skin regions of the infected person or to other persons entirely. As with wart therapies, molluscum lesion treatments can include mechanical means, such as, for example, curettage, surgical excision, laser surgery, and cryotherapy (e.g. with liquid nitrogen or nitrous oxide cryogen). Treatment options also include chemical and immunological methods similar to those used in the treatment of warts, including topical application of podophyllin, cantharidin, iodine with salicylic acid, tretinoin, potassium hydroxide, imiquimod and cidofovir. The treatments available for molluscum contagiosum can be expensive, and they may not be uniformly successful in all patients, particularly those with impaired immune function. In addition, at least one standard treatment for molluscum contagiosum—antharidin—is not available in the U.S. but must be purchased from a foreign source. Another disadvantage, especially with child patients, is that many of the current treatments require multiple visits and/or applications, making it difficult and impractical for families to carryout therapies. Also, cryotherapy and topical application of cantharidin—both commonly used treatments for lesions—can cause painful blistering. Significantly, as with warts, the known available therapies have not been found to be uniformly effective in treating molluscum lesions.

Accordingly, new and improved therapies for the treatment of viral-induced skin lesions, such as, warts and molluscum contagiosum, which are less expensive, more effective, pain-free, and simple to administer would be an advance in the art.

### SUMMARY OF THE INVENTION

The present invention generally relates to compositions comprising mixtures of papain and bromelain, combined with a pharmaceutically acceptable carrier, for treating viral-induced skin lesions, especially including, warts and molluscum contagiosum. The present invention further relates to methods of treating viral-induced skin lesions, especially including, warts and molluscum contagiosum, by administering to a subject in need thereof a therapeutically effective amount of papain and bromelain, preferably directly to the warts or molluscum contagiosum lesions of interest. The papain and bromelain can be administered together as a single composition or they can be separately administered. Administration of the papain and bromelain are preferably administered at or substantially at the same time, i.e. co-administered.

In one aspect, the present invention provides a composition for treating a subject having a viral-induced lesion resulting from a viral infection, where the composition includes a mixture of papain and bromelain and a pharmaceutically acceptable excipient. In certain aspects, the viral-induced lesions treatable by the compositions and methods of the invention are warts. The warts can be genital or non-genital (e.g. common, periungal, flat, filiform, or plantar) warts. In other aspects, the viral-induced lesions treatable by the compositions and methods of the invention are lesions of molluscum contagiosum.

The subjects treatable by the inventive compositions and methods in preferred aspects are humans. In certain aspects, the subjects are children. In certain other aspects, the subjects are immunocompromised persons.

In another aspect, the present invention provides a composition of papain at between about 25-30% and bromelain at between about 5-15% of the composition by weight.

In yet another aspect, the invention provides a composition of papain at between about 25-35% and bromelain at between about 5-15% of the composition by weight.

In a further aspect, the invention provides a composition of papain at between about 20-30% and bromelain at between about 5-10% of the composition by weight.

In still another aspect, the invention provides a composition of papain at about 30% and bromelain at about 10% of the composition by weight.

In a still further aspect, the invention provides a composition of papain at about 25% and bromelain at about 5% of the composition by weight.

The invention also provides in another aspect a composition of papain and bromelain present in a ratio of papain:bromelain of about 10:1, 9:1, 8:1, 7:1, 6:1, 5:1, 4:1, 3:1, 2:1, or 1:1. In another embodiment, the bromelain and papain are present in a ration of bromelain:papain of about 10:1, 9:1, 8:1, 7:1, 6:1, 5:1, 4:1, 3:1, 2:1, or 1:1.

In yet another aspect, the compositions of the present invention include one or more pharmaceutically acceptable excipients is a lecithin organogel base.

In still another embodiment, the present invention relates to a composition of papain and bromelain formulated as a dermatologically-acceptable cream, ointment, salve, lotion, cerate, balm, sunscreen, oil, solution, or the like.

The present invention, in another embodiment, relates to a method for treating a virus-induce lesion resulting from a viral infection comprising administering papain and bromelain directly to the virus-induce lesion in a subject in need thereof. Such virus-induce lesions are in certain aspects warts or molluscum contagiosum lesions.

In another aspect, the invention method is effective in treating non-genital warts, which can include common warts, periungual warts, flat warts, filiform warts, or plantar warts.

In certain aspects, the subjects treatable by the methods of the invention include humans. In other aspects, the subjects are children. In certain other aspect, the subjects are immunocompromised, e.g. HIV-infected persons.

In yet another aspect of the invention, the inventive method relates to administering papain and bromelain in a ratio of about 10:1, 9:1, 8:1, 7:1, 6:1, 5:1, 4:1, 3:1, 2:1, or 1:1. In another embodiment, the bromelain and papain are present in a ration of bromelain:papain of about 10:1, 9:1, 8:1, 7:1, 6:1, 5:1, 4:1, 3:1, 2:1, or 1:1.

In other aspects, the methods of the invention relate to administering papain and bromelain together as a composition. Still other aspects relate to co-administering papain and bromelain as separately prepared formulations.

In aspects involving administering compositions of papain and bromelain, the composition in one embodiment comprises papain at about between 25-35% (w/v) and the bromelain at about between 5-15% (w/v) of the composition. In another embodiment, the composition comprises papain at about 30% (w/v) and the bromelain at about 10% (w/v) of the composition. In yet another embodiment, the composition comprises papain at about between 20-30% (w/v) and the bromelain at about between 5-10% (w/v) of the composition. In a still further embodiment, the composition comprises papain at about 25% (w/v) and the bromelain at about 5% (w/v) of the composition.

In another aspect, the methods of the invention relate to administering compositions of papain and/or bromelain further including one or more pharmaceutically acceptable carriers, such as, for example, lecithin organogel base.

In yet another aspect, the methods of the invention relate to administering compositions of papain and/or bromelain in the form of a dermatologically-acceptable cream, ointment, salve, lotion, cerate, balm, sunscreen, oil, or solution. Administration in certain embodiments can be by topical administration, e.g. by applying a cream or lotion. In other embodiments, administration can be directly injecting the papain and bromelain into the wart or molluscum contagiosum lesion. In further embodiments, the papain and/or bromelain can be administered by a transdermal patch.

The present invention further contemplates carrying out the method of papain and/or bromelain administration in combination with a second therapy aimed at treating the virus-induce lesion. The second therapy in some aspects of the invention can be co-administered, i.e. at or about the same time, as the method of administering papain and bromelain. In other aspects, the second therapy can be administered before or after the method of administering papain and bromelain.

In yet a further aspect, the second therapy relates to mechanical eradication of the virus-induce lesion, e.g. surgery, excision, curettage, laser, cryotherapy, electrocautery, or electrodesiccation. In other aspects, the second therapy relates to chemical treatments aimed at reducing or eradicating the lesion, e.g. acid treatments, such as, trichloroacetic acid, bichloroacetic acid, or salicylic acid, and chemotherapies, such as Retin-A. In still further aspects, the second therapy relates to the administration to the subject in need a therapy that is aimed at boosting or strengthening the immune system, in particular, in a manner that prevents or minimizes re-infection of the underlying viral infection. Such immune-boosting second therapies can include administering antigens, such as, Candida or mumps antigen, interferons, such as, interferon alfa-2b, interferon alfa-N3, or compounds, such as, imiquimod or cimetidine.

It is to be understood that both the foregoing general description and the following detailed description are exemplary and are intended to provide further explanation of the invention claimed. It is also to be understood that features of each embodiment can be incorporated into other embodiments, and that optional features described in connection with one embodiment in accordance with the invention can be incorporated into other embodiments in accordance with the invention.

The accompanying drawings, which are incorporated in and constitute part of this specification, are included to illustrate and provide a further understanding of the method and system of the invention.

### BRIEF DESCRIPTION OF THE DRAWINGS

So that those having ordinary skill in the art to which the subject invention pertains will more readily understand how to make and use the compositions and methods as described herein, preferred embodiments thereof will be described in detail herein below, with reference to the drawings, wherein:

FIG. 1 depicts a plantar wart on the foot of a patient before (A) and after (B) treatment with a papain/bromelain composition of the invention having 30% papain (w/v) and 10% bromelain (w/v).

FIG. 2 depicts a plantar wart on the foot of a patient before (A) and after (B) treatment with the papain/bromelain composition of the invention having 30% papain (w/v) and 10% bromelain (w/v).

FIG. 3 depicts a wart on the finger of a patient before (A) and after (B) treatment with the papain/bromelain composition of the invention having 30% papain (w/v) and 10% bromelain (w/v).

### ENABLING DESCRIPTION OF PREFERRED EMBODIMENTS

The present inventors have unexpectedly found for the first time that the administration of papain and bromelain directly to viral-induced skin lesions, especially including warts and molluscum contagiosum lesions, are effective in treating the lesions. Accordingly, in one aspect, the present invention is directed to new and useful compositions comprising mixtures of papain and bromelain, together with a pharmaceutically acceptable carrier, such as, for example, lecithin organogel, for application to viral lesions, such as, for example, warts and molluscum lesions. In other embodiments, the papain and bromelain can be formulated as separate compositions, and applied to the lesions of interest by co-administration.

For the purposes of the present invention, the following terms are defined.

Definitions

The meanings of the following terms are not intended to be inconsistent with any meaning of the terms that would be known to one of ordinary skill in the art to which this invention pertains.

As used herein, the term “dermatologically-acceptable,” means that the compositions and/or compounds thereof so described are suitable for use in contact with skin without undue toxicity, incompatibility, instability, allergic response, and the like.

As used herein, the phrase “therapeutically effective amount” means an amount of a compound or composition of the invention sufficient to treat, i.e. reduce, ameliorate or eradicate, at least one symptom associated with a treatable viral-induced lesion of the invention, e.g. warts or molluscum contagiosum lesions. It is preferred that the therapeutically effective amount is of a dose that is also low enough to avoid serious side effects, i.e., to provide a reasonable benefit to risk ratio, within the scope of sound judgment of the skilled artisan.

The term “subject” as used herein is a warm-blooded mammal and includes, for instance, humans, and any mammal that is capable of developing a wart or a molluscum contagiosum lesion from a viral infection of the skin or epidermis.

As used herein, the phrase “subject in need thereof” refers to a subject, who has at least one viral-induced lesion, especially including, a wart or a molluscum contagiosum lesion. Alternatively, a subject in need thereof may refer to a mammal, including especially a human, who is at risk of developing a viral-induced lesion. For example, a subject at risk may be a sexual partner of another who has a wart or molluscum contagiosum in the genital region. In another example, a person at risk may be a person who has had skin-to-skin contact with a lesion, e.g. a wart or a molluscum contagiosum lesion, of another.

As used herein, the phrase “co-administration” or the like refers to the administration of the papain and bromelain of the invention at the same time or substantially the same time. The papain and bromelain can be co-administered as a single composition comprising a mixture of papain and bromelain. Co-administration of papain and bromelain can also include where papain and bromelain are formulated separately, e.g. a papain cream and a bromelain solution. At substantially the same time encompasses any useful co-administration regimen that would be determinable by one of ordinary skill in the art without undue experimentation, and may include, for example, administering papain once, twice, thrice, or more a day, followed by the administration of bromelain seconds, minutes or hours later after one or each papain administration. At the minimum, co-administration is meant to encompass any pattern, timing, or administrative schedule of papain and bromelain administration such that both papain and bromelain are present or in contact with the lesion undergoing treatment.

The term co-administration can also refer to the co-administration of papain and bromelain of the invention together with, i.e. at the same time or substantially the same time, as a standard therapy for treating lesions of the invention. Such standard therapies include any mechanical, chemical or immunological method known at the time of the invention for treating lesions of the invention.

The term “obtaining” as in “obtaining papain or bromelain” is intended to include purchasing, synthesizing, preparing by biological means, e.g. genetic engineering, or otherwise acquiring the components and compositions of the invention.

As used herein, the phrase “pharmaceutically acceptable carrier,” is meant to refer to any typically used non-active ingredient in any type of formulation (e.g. cream, oil, or lotion) of the present invention. For example, pharmaceutically acceptable carriers can be any non-active ingredient (i.e. excluding, at least, papain and bromelain) that can be contained in a dermatologically-acceptable cream, ointment, salve, lotion, cerate, balm, sunscreen, oil, solution or the like. For example, in one embodiment, the pharmaceutically acceptable carrier is lecithin organogel. Additional art recognized pharmaceutically acceptable carriers suitable for administering compounds of the present invention to mammals, include liquid or solid fillers, diluents, excipients, solvents or encapsulating materials, involved in carrying or transporting the subject active ingredients to the lesions under treatment. Each carrier must be “acceptable” in the sense of being compatible with the other ingredients of the formulation and not injurious to the patient.

As used herein, the term “treating,” as in a method for treating a viral-induced lesion of the invention (e.g. a wart or molluscum lesion), is meant to refer to reducing, ameliorating or eradicating the lesion. Such reducing, ameliorating or eradicating can occur with respect to a single lesion (e.g. wart) or multiple lesions (e.g. a cluster of warts or molluscum contagiosum legions) and on any bodily location, preferably any non-genital bodily location.

As used herein, the phrase “viral-induced lesion,” or alternatively, “viral-induced skin or epidermal lesion,” refers to a condition resulting from a viral infection of the skin. Viral-induced lesions include, for example, warts, which are caused by papillomavirus (e.g. human papillomavirus (HPV)), and molluscum contagiosum lesions, which are caused by molluscum contagiosum virus (MCV). The lesions can occur on a bodily location, preferably any non-genital bodily location.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Viral-Induced Lesions of the Invention

The present invention contemplates treating any viral-induced lesion capable of being treated by the compositions of the present invention, including especially warts and molluscum contagiosum lesions.

In one preferred embodiment, the viral-induced lesions are warts caused by human papillomavirus (HPV).

In embodiments involving treating warts, no limitation is placed as to the particular genetic variant of HPV associated with the lesion. Further, no limitation is placed on the type or physical characteristics (e.g. size, pattern, shape, thickness, location on the body, hardness or softness, color, etc.) of the particular lesion(s) to be treated. It will be appreciated that a variety of distinct genetic variants of HPV are known, including over 60 types or subtypes of HPV (De Villiers et al., “Classification of papillomaviruses,” Virology. 2004 Jun. 20; 324(1):17-27, incorporated herein by reference).

Papillomaviruses infect a wide variety of different species of animals, including humans. Infection is typically characterized by the induction of benign epithelial and fibro-epithelial lesions, or warts at the site of infection. Each species of vertebrate is infected by a species-specific set of papillomavirus, itself comprising a plurality of different papillomavirus types. For example, more than sixty different human papillomavirus (HPV) genotypes have been isolated. Papillomaviruses are highly species-specific infective agents. For example, canine and rabbit papillomaviruses cannot induce papillomas in heterologous species such as humans. Neutralizing immunity to infection against one papillomavirus type generally does not confer immunity against another type, even when the types infect a homologous species.

Papillomaviruses are non-enveloped DNA viruses that induce hyperproliferative lesions of the epithelia. The papillomaviruses are widespread in nature and have been identified in higher vertebrates. Most animal papillomaviruses are associated with purely epithelial proliferative lesions, and most lesions in animals are cutaneous. In humans there are more than 60 types of papillomavirus that have been identified and they have been catalogued by site of infection: cutaneous epithelium and mucosal epithelium (oral and genital mucosa).

The life cycle of papillomavirus is closely coupled to differentiation of keratinocytes (major cell type making up the epidermis). Infection is believed to occur at a site of tissue disruption in the basal epithelium. Unlike normal cells, the cellular DNA replication machinery is maintained as the cell undergoes vertical differentiation. As the infected cells undergo progressive differentiation the viral genome copy number and viral gene expression in turn increase, with the eventual late gene expression and virion assembly in terminally differentiated keratinocytes and the release of viral particles.

The coding strands for each of the papillomavirus contain approximately ten designated translational open reading frames (ORFs) that have been classified as either early ORFs or late ORFs based on their location in the genome. E1 to E8 are expressed early in the viral replication cycle, and two late genes (L1 and L2) encode the major and minor capsid proteins, respectively. The E1 and E2 gene products function in viral DNA replication, whereas E5, E6 and E7 are expressed in connection with host cell proliferation. The L1 and L2 gene products are involved in virion structure. The function of the E3 and E8 gene products is uncertain at present.

In another preferred embodiment, the viral-induced lesions are lesions of molluscum contagiousum caused by the molluscum contagiosum virus (MCV).

In embodiments involving treating molluscum lesions, no limitation is placed as to the particular genetic variant of MCV associated with the lesion. Further, no limitation is placed on the type or physical characteristics (e.g. size, pattern, shape, thickness, location on the body, hardness or softness, color, etc.) of the particular lesion(s) to be treated. It will be appreciated that a variety of distinct genetic variants of MCV are known, including at least 3 types or subtypes of MCV (MCV I, MCV II, and MCV III) (Porter et al., “Characterisation by restriction mapping of three subtypes of molluscum contagiosum virus,” J Med Virol. 1992 September; 38(1):1-6, which is incorporated herein by reference).

Molluscum contagiosum, a cutaneous and mucosal lesion caused by a molluscipox virus (MV) or molluscum contagiosum virus (MCV), is transmitted primarily through direct skin contact with an infected individual. Fomites have been suggested as another source of infection, with molluscum contagiosum reportedly acquired from bath towels, tattoo instruments, and in beauty parlors and Turkish baths (Postlethwaite R. Molluscum contagiosum: A review. Arch Environ Health 1970; 21: 432-452). The average incubation time is between 2 and 7 weeks with a range extending out to 6 months. Infection with the virus causes hyperplasia and hypertrophy of the epidermis (Billstein S A. Mattaliano V J Jr. The “nuisance” sexually transmitted diseases: Molluscum contagiosum, scabies, and crab lice. Med Clin North Am 1990; 74: 1487-1505). Free virus cores have been found in all layers of the epidermis. So-called viral factories are located in the malpighian and granular cell layers (Id.). The molluscum bodies contain large numbers of maturing virions. These are contained intracellularly in a collagen-lipid-rich saclike structure that is thought to deter immunological recognition by the host (Nakamura J, Muraki Y, Yamada M, Hatano Y, Nii S. J Med Virol 1995; 46(4):339-48). Rupture and discharge of the infectious virus-packed cells occur in the center of the lesion. MCV induces a benign lesion instead of the usual necrotic pox lesion associated with other poxviruses (Diven D G, An overview of poxviruses, J AM Acad Dermatol 2001; 44:1-14).

More in particular, MCV typically produces multiple umbilicated lesions. The individual lesions are discrete, smooth, and dome shaped. They are generally skin colored with an opalescent character. The central depression or umbilication contains a white, waxy curdlike core. The size of the papule is variable, depending upon the stage of development, usually averaging 2-6 mm. Papules may exceed 1 cm in size in immunosuppressed hosts. The papules may become inflamed spontaneously or after trauma and present atypically in size, shape, and color. The lesions are often grouped in small areas but may also become widely disseminated.

Any cutaneous surface may be involved, but favored sites include the axillae, the antecubital and popliteal fossae, and the crural folds. For the purposes of the present invention, any skin region capable of being infected by MCV can be treated by the herewith described methods and compositions. Rarely, MCV lesions occur in the mouth or conjunctivae (Whitaker S B, Wiefand S E, Budnick S D. Inraoral molluscum contagiosum. Oral Surg Oral Med Oral Pathol. 1991; 72: 334-336; Ingraham H J, Schoenleber D B. Epibulbar molluscum contagiousm. Am J opthalmol 1998; 125:394-396; Vannas S, Lapinleimu K. Molluscum contagiosum of the skin, caruncle, and conjunctiva. Acta Opthalmol 1967; 45:314-321). Autoinoculation is common. Children usually acquire molluscum nonsexually at both genital and nongenital areas. MCV in adults affects the groin, genital area, thighs, and lower abdomen and other places on the body and is often acquired sexually. Around 10% of cases develop an eczematous dermatitis around the lesions, but this disappears as the infection resolves (De Oreo G A, Johnson H H, Binkley G W. An eczematous reaction associated with molluscum contagiosum. Arch Dermatol 1956; 74: 344-8). Patients with atopic dermatitis can have a disseminated eruption. Eruptions in immunocompromised individuals are very resistant to treatment (Schwartz J J. Myskowski P L: Molluscum contagiosum in patients with human immunodeficiency virus infection. J Am Acad Dermatol 1992; 27:583; Cotton D W, Cooper C, Barrett D F, Leppard B J. Severe atypical molluscum contagiosum infection in an immunocompromised host. Br J Dermatol 1987; 116:871-876).

Histologically, molluscum contagiosum exhibits intraepidermal lobules with central cellular and viral debris. In the basal layer, enlarged basophilic nuclei and mitotic figures are seen. Progressing upward, the cells show cytoplasmic vacuolization and then eosinophilic globules. The nucleus becomes compressed at the level of the granular cell layer, and the molluscum bodies lose their internal structural markings. Undisrupted lesions show an absence of inflammation, but dermal changes can include an infiltrate that is lymphohistiocytic, neutrophilic, or granulomatous. The latter has been seen in solitary lesions. Antibody to MCV by indirect immunofluorescence has been found in 69% of patients with visible lesions (Shirodaria P V, Observations on the antibody responses in molluscum contagiosum, Br J Dermatol 1997; 96:29-34). Polymerase chain reaction can detect MCV in skin lesions (Thompson C H. Identification and typing of molluscum contagiosum virus in clinical specimens by polymerase chain reaction. J Med Virol 1997; 53:205-211).

The clinical appearance of molluscum contagiosum is in most cases diagnostic. Though molluscum cannot be cultured in the laboratory, histological examination of a curetted or biopsied lesion can also aid in the diagnosis in cases that are not clinically obvious. The thick white central core can be expressed and smeared on a slide and left unstained or stained with Geimsa, Gram, Wright, or Papanicolaou stains to demonstrate the large brick-shaped inclusion bodies. Electron microscopy has also been used to demonstrate the poxvirus structures. Immunohistochemical methods using a polyclonal antibody allows recognition of molluscum contagiosum in fixed tissue (Penneys N J, Matsuo S, Mogollon R. The identification of molluscum infection of immunohistochemical means. J Cutan Pathol 1986; 13:97-101). In-situ hybridization for MCV DNA has also been utilized (Thompson C H, Biggs I M, DeZwart-Steffe R T. Detection of molluscum contagiosum virus DNA by in-situ hybridization. Pathology 1990; 22:181-186). Molluscum contagiosum lesions can be differentiated from verruca vulgaris, condyloma accuminata, varicella, herpes simplex, papillomas, epitheliomas, pyoderma, cutaneuos cyptococcosis, epidermal inclusion cyst, basal cell carcinoma, papular granuloma annulare, keratoacanthoma, lichen planus, and syringoma or other adenexal tumors.

Although warts and molluscum lesions are preferably treated by the present invention, any lesion capable of being treated by the instant methods and compositions is contemplated by the present invention. Such additionally treatable lesions and conditions will be determinable by one of ordinary skill in the art without undue experimentation.

Active Ingredients

The present invention, in a further embodiment, contemplates compositions comprising active ingredients papain and/or bromelain.

Papain is a proteolytic (protein-digesting) enzyme derived from the fruit and leaves of the papaya plant (Carica papaya), which contains both papain and chymopapain. More specifically, papain is a cysteine protease consisting of 212 amino acid mature polypeptide which is stabilised by 3 disulfide bridges, and which is expressed as a 345 precursor polypeptide (Cohen et al., Cloning and sequencing of papain-encoding cDNA, Gene 48: 219-227). Its three-dimensional structure has been determined to consist of 2 distinct structural domains with a cleft between them. This cleft contains the active site, which contains a catalytic triad that has been likened to that of chymotrypsin. Its catalytic triad is made up of 3 amino acids—cysteine-25 (from which it gets its classification), histidine-159, and asparagine-158. The mechanism by which it breaks peptide bonds involves deprotonation of Cys-25 by His-159. Asn-158 helps to orient the imidazole ring of His-159 to allow this deprotonation to take place. Cys-25 then performs a nucleophilic attack on the carbonyl carbon of a peptide backbone. This frees the amino terminal of the peptide, and forms a covalent acyl-enzyme intermediate. The enzyme is then deacylated by a water molecule, and releases the carboxy terminal portion of the peptide. In immunology, papain is known to cleave the Fc (crystallisable) portion of immunoglobulins (antibodies) from the Fab (antigen-binding) portion.

Prior to the present invention, papain had a variety of well-known uses. In one aspect, papain has been used for many years in the tenderizing in breaking down meat. For example, it is sold as a component in powdered meat tenderizer available in most supermarkets. Papain can be made into a paste with water as a home remedy treatment for jellyfish, bee, yellow jacket (wasps) stings and possibly stingray wounds, breaking down the protein toxins in the venom.

In cell biology, papain can be used to help dissociate cells in the first step of cell culture preparations. It is also used as an ingredient in various enzymatic debriding preparations, e.g. Accuzyme. These are used in the care of some chronic wounds to clean up dead tissue. It can also be found as ingredient in some toothpastes or mints as a tooth-whitener agent. It's whitening effect in toothpastes and mints, however, is minimal, because the papain is present in low concentrations, and will be quickly diluted by saliva. It has also been described as a component of a compound for treatment of athlete's foot (a fungal infection). An injectable purified form of chymopapain has been used to treat herniated discs in the spine.

Any means for obtaining papain for use in the present invention is contemplated. Papain can be obtained from natural sources or prepared by recombinant means.

For example, papain can be produced as a crude, dried material by collecting the latex from the fruit of the papaya tree. The latex is collected after scoring the neck of the fruit where it may either dry on the fruit or drip into a container. This latex is then further dried. Any suitable purification step or set of steps can then be carried out to remove contaminating substances and then stored in any form, such as a liquid or powder. The papaya from which the papain is obtained can be any naturally-occurring papaya plant, or alternatively, the papaya plant can be a genetically modified variant.

In another aspect, papain can be obtained by recombinant means, for example, by cloning and expressing the gene or coding region thereof in a recombinant expression system, e.g. by expression in a bacterial or eukarotic expression system. Both the gene encoding papain, and the amino acid sequence of papain of papaya are well-known in the art (e.g. Mitchel et al., The complete amino acid sequence of papain, The Journal of Biological Chemistry, 1970, Vol. 245, No. 14, pp: 3485-3492; Cohen et al., Cloning and sequencing of papain-encoding cDNA, Gene 48: 219-227; and Lee et al., Complete amino acid sequence of ananain and a comparison with stem bromelain and other plant cysteine proteases [including papain], Biochem J. (1997), 327: 199-202, each of which are incorporated herein by reference). In one aspect, the papain of the present invention has the amino acid sequence of SEQ ID NO: 1:

IPEYVDWRQK GAVTPVKNQG SCGSCWAFSA VVTIEGIIKI
RTGNLNEYSE QELLDCDRRS YGCNGGYPWS ALQLVAQYGI
HYRNTYPYEG VQRYCRSREK GPYAAKTDGV RQVQPYNEGA
LLYSIANQPV SVVLEAAGKD FQLYRGGIFV GPCGNKVDHA
VAAVGYGPNY ILIKNSWGTG WGENGYIRIK RGTGNSYGVC
GLYTSSFYPV KN

(Mitchel et al., The complete amino acid sequence of papain, The Journal of Biological Chemistry, 1970, Vol. 245, No. 14, pp: 3485-349, incorporated herein by reference). SEQ ID NO: 1 is exemplary and the papain of the present invention is not meant to be limited thereto.

Methods of recombinant DNA technology for obtaining papain by recombinant means will be well-known to those of ordinary skill in the art. Such methods can be found, for example, in Molecular Cloning: A Laboratory Manual, Cold Spring Harbor Laboratory Press; 3^{rd} Labmn edition (2001), which is incorporated herein by reference.

The present invention is not meant to be restricted to any particular papain amino acid sequence (or any encoding nucleotide sequence) and is meant to encompass any papain amino acid sequence substantially homologous or functionally equivalent to SEQ ID NO: 1, or a functionally equivalent fragment thereof. The term “substantially homologous” will be well understood by one skilled in the art who would easily be able to determine whether or not two sequences were substantially homologous. However, in general, amino acid sequences can be described as substantially homologous when they have at least 40% homology (i.e. percent sequence identity between aligned sequences) although, for the purpose of the present invention, it is preferable for a sequence to have at least 50%, 60%, 70%, 80%, 90% or 95% or even 99% homology to the amino acid sequence of SEQ ID NO 1. It will be appreciated that sequences homologous to SEQ ID NO: 1 can be obtained in nature (e.g. naturally obtained papaya, or naturally occurring papaya variant) or through other means, such as, genetic engineering, such as by mutagenesis of genes isolated from nature.

Bromelain is a mixture of various moieties derived from the stem of the pineapple (Ananas comosus). It contains at least two proteolytic enzymes but also non-proteolytic enzymes, including an acid phosphatase and a peroxidase; it may also contain amylase and cellulase activity. Any means for obtaining bromelain for use in the present invention is contemplated. For example, bromelain may be extracted from pineapple juice through precipitation with acetone and also with ammonium sulfide. The bromelain may also be obtained from any suitable commercial source. The principle component of commercially available bromelain is a glycoprotein proteolytic enzyme with a molecular weight of approximately 33,000 Daltons. It has been used for tenderizing meat. It has been reported to have anti-inflammatory activity, to inhibit platelet aggregation, and to be a selective prostaglandin inhibitor. Among the conditions for which it has been used are arthritis, neuralgias, sprains, bursitis, myositis, eczema, inflammation due to psoriasis, and infections. Bromelain has been used as a digestive aid, and as a cleansing agent for the skin. It has also been described for treatment of wounds to promote healing.

The known proteolytic enzymes of bromelain and papain share a high degree of amino acid sequence homology around the active centre, and evidence suggests that bromelain and papain use the same catalytic mechanism. Bromelain differs from papain, however, in having a different specificity of cleavage. In addition, the known proteolytic enzymes of bromelain are glycoproteins, whereas papain is a simple protein. Bromelain is reviewed by Taussig et al, J. Ethnopharmacol. 22 191-203 (1988), which is incorporated herein by reference.

Bromelain has been used widely for a variety of purposes. In one example, bromelain is previously used as an adjunct in the treatment of soft tissue inflammation and oedema associated with trauma and surgery. Bromelain is available in various countries under the trademarks ANANASE FORTE, ANANASE, EXTRANASE, PROTEOLIS, RESOLVIT, ROGORIN, BROMASE and TRAUMANASE. In clinical use over a period of more than 30 years, there have been few reports of significant undesirable effects. Stem bromelain is a crude mixture of many colloids (including proteins, carbohydrates and mucopolysaccharides), inorganic salts and simpler organic materials which are precipitated from the juice of the pineapple stem by acetone. Protein generally constitutes about 50% of the total weight of the dried precipitate, inorganic materials, principally cations (calcium, magnesium, potassium, copper and iron) generally make up to 10-15% of the total weight. The balance is assumed to be complex carbohydrate materials of the nature of polyuronides and glycosides. Crude stem bromelain demonstrates considerable enzymatic activity including proteolytic and acid phosphatase activities and lesser peroxidase, amylase and pectin esterase activities.

Significantly, while papain and bromelain are found in the art, it is believed that the present inventors were the first to conceive of a method of treating viral-induced lesions use a mixture of papain and bromelain.

Stem bromelain protease has been described by Ritonja et al (Febs Letters, 247, 419-424 (1989)) and has the amino acid sequence set out below (SEQ ID NO: 2):

Ala Val Pro Gln Ser Ile Asp Trp Arg Asp Tyr Gly 1 5 10 Ala Val Thr Ser Val Lys Asn Gln Asn Pro Cys Gly 15 20 Ala Cys Trp Ala Phe Ala Ala Ile Ala Thr Val Glu 25 30 35 Ser Ile Tyr Lys Ile Lys Lys Gly Ile Leu Glu Pro 40 45 Leu Ser Glu Gln Gln Val Leu Asp Cys Ala Lys Gly 50 55 60 Tyr Gly Cys Lys Gly Gly Trp Glu Phe Arg Ala Phe 65 70 Glu Phe Ile Ile Ser Asn Lys Gly Val Ala Ser Gly 75 80 Ala Ile Tyr Pro Tyr Lys Ala Ala Lys Gly Thr Cys 85 90 95 Lys Thr Asp Gly Val Pro Asn Ser Ala Tyr Ile Thr 100 105 Gly Tyr Ala Arg Val Pro Arg Asn Asn Glu Ser Ser 110 115 120 Met Met Tyr Ala Val Ser Lys Gln Pro Ile Thr Val 125 130 Ala Val Ala Asp Ala Asn Ala Asn Phe Gln Tyr Tyr 135 140 Lys Ser Gly Val Phe Asn Gly Pro Cys Gly Thr Ser 145 150 155 Leu Asn His Ala Val Thr Ala Ile Gly Tyr Gly Gln 160 165 Asp Ser Ile Ile Tyr Pro Lys Lys Trp Gly Ala Lys 170 175 180 Trp Gly Glu Ala Gly Tyr Ile Arg Met Ala Arg Asp 185 190 Val Ser Ser Ser Ser Gly Ile Cys Gly Ile Ala Ile 195 200 Asp Pro Leu Tyr Pro Thr Leu Glu Glu 205 210. SEQ ID NO: 2 is exemplary and the bromelain of the present invention is not meant to be limited thereto.

In a further aspect of the invention, there is provided the use of stem bromelain protease having the amino acid sequence of SEQ ID NO: 2 or a sequence substantially homologous or functionally equivalent thereto for the use in the compositions of the invention. In general, amino acid sequences can be described as substantially homologous when they have at least 40% homology although, for the purpose of the present invention, it is preferable for a sequence to have at least 50%, 60%, 70%, 80%, 90% or 95% or even 99% homology to the amino acid sequence of SEQ ID NO 2, or fragment thereof. In addition, the residues which are compared need not be in exactly the same positions in two sequences which are substantially homologous but rather, one of the sequences may have various inserted or deleted amino acid residues or regions with respect to the sequence with which it is compared.

The following U.S. patents disclose various way of isolating bromelain: U.S. Pat. Nos. 6,335,427, 6,095,307, 5,928,640, 5,824,305, 5,767,066, 5,441,740, 5,387,517, 4,521,254, and 4,286,064, each of which are incorporated herein by reference.

Pharmaceutical Compositions

In another embodiment, the present invention provides a pharmaceutically acceptable composition comprising the papain and/or bromelain compounds of the invention.

The expression “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Pharmaceutically acceptable includes those compositions that are also dermatologically acceptable, such as creams, lotions, gels, oils, and the like comprising papain and/or bromelain.

Generally, no limitation is placed on the particular form and nature of the pharmaceutical compositions, i.e. the formulations, of the present invention. Preferably, however, the formulations of the invention relate to those that can be directly administered to a lesion of interest. Such formulations generally include topical formulations, such as, but not limited to, creams, lotions, oils, sunscreens, and wettable powders (i.e. where powder form of active ingredients is wetted, dissolved, emulsified, or generally transferred into a liquid or aqueous environment), emulsions, and the like. The formulation can also be prepared as a pharmaceutically acceptable solution for direct injection into a lesion. In another embodiment, the formulation can be prepared to be deliverable transdermally, e.g. a transdermal patch, bandage, or the like.

Any of the contemplated formulations of the invention can be prepared to contain one or both of the active ingredients. For example, a cream comprising a suitable dose of papain can be prepared and applied to a patient in need together with a cream comprising a suitable dose of bromelain. The papain and bromelain can also be formulated separately as the same or different formulation types, e.g. papain could be formulated as a cream, whereas bromelain could be formulated as an oil, etc. In a preferred embodiment, the papain and bromelain are co-administered, as either a single combined formulation, or two or more separate formulations of papain and bromelain of either the same type (e.g. each are creams) or different types (e.g. one is a cream and one is an oil).

Preferably, the active ingredients, papain and bromelain, are formulated with one or more pharmaceutically acceptable carriers. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical and/or dermatologically-acceptable formulations. In one preferred embodiment, the pharmaceutically acceptable carrier is lecithin organogel.

Generally, the papain and bromelain compositions described herein may be formulated for topical application with pharmaceutically acceptable carriers using methods well known in the cosmetic and pharmaceutical arts, including gels, creams, ointments, emulsions, dispersions, salves, pastes, lotions and the like. These formulations may additionally comprise one or more emulsifiers, humectants (e.g., glycerin or glycerol, sorbitol, and the other known polyols), skin conditioning agents (e.g., propylene glycol, sweet almond oil, apricot kernel oil), surfactants (e.g. ceteth-20), colorants such as staining dyes and pigments (e.g, calcium, barium and aluminum lakes, iron oxides, titanium dioxide and mica), antioxidants (i.e., ascorbic acid, tocopherols, ascorbyl palmitate, thiodipropionic acid), viscosity-enhancing agents (e.g., cetearyl alcohol, polyethylene glycol), vitamins, minerals, emollients, skin conditioning agents, biological additives (e.g. botanicals or herbals), sunscreens (e.g. octyl methoxycinnamate, butyl methoxydibenzolylmethane, oxybenzone), pH adjusters, solvents, germicides (e.g., antibiotics, Tricolsan), preservatives (e.g., BHT, methylparaben, ethylparaben, propylparaben, butylparaben) and fragrances (e.g., strawberry extract, mangifera indica). It will be appreciated by those of skill in the art that particular compounds may be properly classified in one, or two or more of the above-listed classifications of compound types.

The compositions may also include one or more biological additives typical of cosmetic and dermatologically acceptable formations, such as botanicals or herbals. As used herein, the term “biological additive” indicates any compound obtained from a natural source, including plants, animals, bacteria and yeast, which has a medicinal or otherwise beneficial effect when topically applied to the skin and which may assist, facilitate, or enhance the action of the papain and bromelain of the invention. Examples of biological additives include oil of Melaleuca alternifolia, oil of Lavandula angustifolia, Echinacea angustifolia extract, Mimosa tenuiflora extract, Hydrocotyl (centella) asiatica extract, gingko biloba extract, oil of Melaleuca alternifolia (tea tree oil), Matricaria chamomila (chamomile) extract, Hypericum perforatum extract, Aloe barbedensis extract, and the like. The biological sources for “biological additive” may also include, but are not limited to the following: Aloe Vera, Aloe Barbedensis; Arnica, Arnica Montana; Bladderwrack (seaweed), Fucus Vesciculosis; Birch, Betula Alba (Pendula); Chamomile, Matricaria Chamomila (Chamomila Recutita); Marsh Mallow, Althea Officinalis; Meadow Sweet, Spirea Ulmaria (Filipendula); Mint/Lemon Balm, Melissa Officinalis; Mimosa, Mimosa Tenuiflora; Myrrh Tincture, Commiphor Myrrha; Neem, Melia Azadirachta; Nettle (stinging), Urtica Dioica; Papaya, Carica Papaya; Propolis (bee glue), Propolis Cera; Raspberry, Rubis Idaeus; Red Poppy, Papaver Rhoeas; Rose Hip (dog rose), Rosa Carima; Rosemary, Rosemarinus Officinalis; Sage, Salvia Officinalis; St. Johns Wort, Hypericum Perforatum; Strawberry, Fragaria Vesca; Thea Sinensis (green tea), Camelia Sinensis; Walnut, Juglans Regia; Witchhazel (dist/extr), Hamamelis Virginiana; Yarrow, Achillea Millefolium; Wild Yam, Dioscorea Villosa; Hawthorn, Crataegus Monogina/Oxyantha; Herma (black/rod), Lawsoma Ehemus; Hops, Humulus Lupulus; Horse Chestnut, Aesculus Hippocastanum; Horse Tail, Equisitum Arvense; Ivy, Hedera Helix; Linden/Lime Tree Blossoms, Tilia Argentea Cordata; Madder, Rubia Tinctorum; Marigold, Calendula Officinalis; Centella Asiatica, Centella Asiatica Urban (hydrocotyl Asiatica); Carrot (roots), Daucus Carota; Comfrey (Allantoine), Symphytum Officinale; Coneflower (Echinacea), Echinacea Angustifolia; Cucumber, Cucumis Sativus (Frucus Cucumis); Fenugreek, Trigonella Foenum Greacum; Gingko, Gingko Biloba; Ginseng, Panax Ginseng; Great Burdock, Radix Bardanea/Arctium Lappa; Tea Tree Oil, Oil of Melaleuca Alternifolia; Colts Foot, Tussilago Farfara; Clover, Trifolium Pratense; Speedwell, Veronica Officinalis.

Further biological additives, along with the biological or medicinal properties of the biological additives described herein and of other known biological additives are known to those of skill in the art. References, including encyclopedias and treatises, known to those of skill in the art, that described such biological additives, along with the biological or medicinal properties of the biological additives described herein, include: Guenther—The Essential Oils, Van Nostrand; Int. Cosmetic Ingredient Dictionary, Vol 1 & 2, C.T.F.A. 1995; Int. Cosmetic Ingredient Handbook, C.T.F.A. 1995; British Herbal Pharmacopoeia, British Herbal Medicine Assoc., 1983; Clinical Applications of Ayurvedic & Chinese Herbs, K. Bone, Phytotherapy Press, 1996; A Handbook of Chinese Healing Herbs, D. Reed, Shambala, Boston, 1995; Echinacea—Nature's Immune Enhancer, S. Foster, Healing Arts Press, Rochester, 1991; Encyclopedia of Herbs, D. Brown, RD Press, 1995; Encyclopedia of Medicinal Plants, A. Chevalier, Dorling Kingers Ley, 1996; L'Angelica—Herbal Extracts; Cosmetochem—Herbasol Extracts. These references are incorporated herein in their entireties.

Emulsifiers contemplated for use include, but are not limited to, monoacyl glycerol, such as glyceryl monoalkanoates, glyceryl monoalkenoates, diacyl 1,2- or 1,3-disubstituted) glycerol, such as glyceryl dialkanoates, glyceryl dialkenoates, polyglyceryl esters, stearic acid, cetyl alcohol, and sorbitan stearate.

In preferred aspects, the formulations of the invention comprising one or more of the above active ingredients, non-active ingredients, emulsifiers and biological additives, should be in of a form and consistency such that the active ingredients of papain and/or bromelain are well-preserved and stable over time, e.g. active ingredients remain stable from the point of manufacture (or mixing by user) to the point of use. In further preferred aspects, it is desirable that the active ingredients of papain and/or bromelain remain substantially homogenously dispersed or dissolved in the particular formulation used such that it is possible to deliver or apply consistent amounts of active ingredients as a function of the amount or the volume of the formulation applied.

In one preferred embodiment, the topical formulation is VANPEN™ Cream (available commercially from e.g. Kenmore Rx Center, New York), a cream comprising urea, edentate disodium 5% solution, stearic acid NF flakes, simethicone USP, ethosy diglycol liquid, squalane NF, polysorbate, cetyl alcohol, glycerin USP, glyceryl monostearate pure, lecithin organogel solution, BHT.

In another embodiment, the papain and bromelain compositions of the present invention are formulated for direct administration by injection into a lesion of interest. Formulations suitable for administration of the inventive compositions by direct injection can include a sterile aqueous preparation of papain and/or bromelain. This aqueous preparation may be formulated according to known methods using suitable dispersing or wetting compounds and suspending compounds. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables.

In general, carrier formulations suitable for oral, subcutaneous, intravenous, intramuscular, etc. administrations can be found in Remington's Pharmaceutical Sciences, Mack Publishing Co., Easton, Pa.

In yet another embodiment, the papain and bromelain compositions of the present invention are formulated for administration by transdermal delivery, e.g. a transdermal patch or bandage or the like. Transdermal delivery technology is well known in the art and any suitable transdermal system and formulation suitable for same is contemplated by the present invention.

It will be appreciated that the skin is made up of several layers with the upper composite layer being the epithelial layer. The outermost layer of the skin is the stratum corneum which has well known barrier properties to prevent molecules and various substances from entering the body and analytes from exiting the body. The stratum corneum is a complex structure of compacted keratinized cell remnants having a thickness of about 10-30 microns. The stratum corneum forms a waterproof membrane to protect the body from invasion by various substances and the outward migration of various compounds. The natural impermeability of the stratum corneum prevents the administration of most agents and other substances through the skin. Numerous methods and devices have been proposed to enhance the permeability of the skin and to increase the diffusion of various drugs through the skin so that the drugs can be utilized by the body. Typically, the delivery of drugs through the skin is enhanced by either increasing the permeability of the skin or increasing the force or energy used to direct the drug through the skin.

Transdermal form of delivery of the inventive compositions can be effected according to methods known in the art. Generally, transdermal delivery involves the use of a transdermal “patch” which allows for slow delivery of compound to a selected skin region, e.g. at or near a wart of molluscum lesion. Although such patches are generally used to provide systemic delivery of compound, in the context of the present invention, such site-directed delivery can be expected to provide increased concentration of compound in selected regions of lesion proliferation.

Examples of transdermal patch delivery systems are provided by U.S. Pat. No. 4,655,766 (fluid-imbibing osmotically driven system), and U.S. Pat. No. 5,004,610 (rate controlled transdermal delivery system), as well as in U.S. Pat. Nos. 3,742,951, 3,742,951, 3,797,494, 3,996,934, 4,031,894, 5,023,252, and 6,096,334, each of which are incorporated in their entireties by reference.

For transdermal delivery, it may be desirable to include permeation enhancing substances, such as fat soluble substances (e.g., aliphatic carboxylic acids, aliphatic alcohols), or water soluble substances (e.g., alkane polyols such as ethylene glycol, 1,3-propane-diol, glycerol, propylene glycol, and the like). In addition, as described in U.S. Pat. No. 5,362,497, a “super water-absorbent resin” may be added to transdermal formulations to further enhance transdermal delivery. Examples of such resins include, but are not limited to, polyacrylates, saponified vinyl acetate-acrylic acid ester copolymers, cross-linked polyvinyl alcohol-maleic anhydride copolymers, saponified polyacrylonitrile graft polymers, starch acrylic acid graft polymers, and the like. Such formulations may be provided as occluded dressings to the region of interest, or may be provided in one or more of the transdermal patch configurations described above.

For delayed release of the active ingredients of the invention, the inventive compositions comprising papain and/or bromelain may be included in a pharmaceutical composition for slow release, such as in microcapsules formed from biocompatible polymers or in liposomal carrier systems according to methods known in the art.

The skilled artisan will appreciate that transdermal delivery can be accompanied by its own side effects, including a potential for skin irritation, arising from the gel or other matrix, from the pharmaceutical itself, or from the interaction of the pharmaceutical with the matrix. Furthermore, a transdermal system preferably should be configured such that the combination of the matrix and the pharmaceutical does not react with or modify the active ingredients, or otherwise render them ineffective, such that the combination provides sufficient diffusion coefficients, such that the delivery system is not adversely affected by expected temperature variations during normal manufacture, transportation, storage and use, such that the gel or other matrix retain the desired viscosity, and such that the active ingredients can be properly dispersed or dissolved in the matrix such that components remain homogenous and do not separate (particularly when more than one compound is included) and the like.

The present invention contemplates other devices and methods known in the art which are designed to facilitate transdermal delivery of agents. One example of a method for increasing the delivery of drugs through the skin include iontophoresis. Iontophoresis generally applies an external electrical field to ionize the drug, thereby increasing the diffusion of the drug through the skin. Iontophoresis can be difficult to control the amount and rate of drug delivery. Under some circumstances, iontophoresis can cause skin damage depending on the extent of ionization, the energy applied to ionize the drug and duration of the treatment.

Sonic energy, and particularly ultrasonic energy, has also been used to increase the diffusion of drugs through the skin. The sonic energy is typically generated by passing an electrical current through a piezoelectric crystal or other suitable electromechanical device. Although numerous efforts to enhance drug delivery using sonic energy have been proposed, the results generally show a low rate of drug delivery.

Another method of delivering drugs through the skin is by forming micropores or cuts through the stratum corneum, e.g. near of on the site of a lesion of the invention. By penetrating the stratum corneum and delivering the drug to the skin in or below the stratum corneum, many drugs can be effectively administered. The devices for penetrating the stratum corneum generally include a plurality of micron size needles or blades having a length to penetrate the stratum corneum without passing completely through the epidermis. Examples of these devices are disclosed in U.S. Pat. No. 5,879,326 to Godshall et al.; U.S. Pat. No. 5,250,023 to Lee et al., and WO 97/48440, each of which are incorporated herein by reference.

Pulsed laser light can also be used to improve transdermal delivery. In this approach, the pulsed laser light is used to ablate the stratum corneum without significant ablation or damage to the underlying epidermis. The compositions of the invention are then applied to the ablated area and allowed to diffuse through the epidermis.

The present invention encompasses any of the above-described systems or approaches for delivering the active ingredients, papain and bromelain, to a lesion of interest, e.g. a wart or a molluscum lesion. Delivery by others routes are also possible and not meant to be excluded from the present invention. The skilled artisan will have within his or her ability to evaluate, choose, and utilize any suitable means of delivering the active ingredients of the invention to a subject in need thereof.

The relative amounts of papain and bromelain in any of the above formulations and compositions can be varied without undue experimentation by a person of ordinary skill in the art. Further, the relative amounts of papain and bromelain can be raised or lowered or their ratios can be changed or adjusted depending on any number of factors, including, for example, the time of day of the particular application (e.g. may want to use a lower dosage of each active ingredient during the daytime, but a higher dosage of each overnight), the point in the course of treatment (e.g. may want to use higher dosages at first, followed by decreased dosages at a later point in treatment), and the size and/or severity of the lesion (e.g. may want to use higher dosage for larger warts or lesions occurring in greater numbers). In addition, the relative amounts of papain and bromelain in the composition of the invention can be raised or lowered as a function of the frequency of administration of the compositions of the invention or the location on the body to be administered (e.g. lower amounts can be applied to sensitive regions, such as, the genital and ophthalmic or ocular regions). Further, the form of the composition, i.e. the type of formulation, can also impact on the dosage used and the frequency of separate administrations (e.g. a slow-release transdermal patch can include a greater amount of active ingredient applied once per treatment, whereas a cream applied three-times daily for two consecutive weeks can have a lower dose).

In one embodiment of the subject invention, the pharmaceutically acceptable composition for the treatment of warts is provided. Preferably, the pharmaceutically acceptable composition comprises papain in a concentration of about between 25% to 35%, and bromelain in a concentration of about between 5% to 15%, and a pharmaceutically acceptable carrier. In another preferred embodiment, the present invention comprises a formulation of papain in a concentration of about 30%, and bromelain in a concentration of about 10%, and a pharmaceutically acceptable carrier. In a further preferred embodiment, the pharmaceutically acceptable carrier is lecithin organogel base.

In another embodiment of the instant invention, the pharmaceutically acceptable composition for the treatment of molluscum contagiosum lesions is provided. Preferably, the pharmaceutically acceptable composition comprises papain in a concentration of about between 20% to 30%, and bromelain in a concentration of about between 5% to 10%, and a pharmaceutically acceptable carrier. In another preferred embodiment, the present invention comprises a formulation of papain in a concentration of about 25%, and bromelain in a concentration of about 5%, and a pharmaceutically acceptable carrier. In a further preferred embodiment, the pharmaceutically acceptable carrier is lecithin organogel base.

The present invention further contemplates a pharmaceutical composition having papain at about 30% and bromelain at about 10% of the weight of the composition. Also contemplated is a pharmaceutical composition having papain at about 25% and the bromelain at about 5% of the weight of the composition.

In certain further embodiments, the papain can be in a concentration of about between 1% to 5%, more preferably about between 5% to 15%, or even about between 10% to 20%, or even more preferably about between 15% to 25%, or still more preferably about between 20% to 30%, or even 35% or 40%, and as much as even about 50% or even more. In certain other embodiments, the bromelain of the invention can be in a concentration of about between 1% to 5%, more preferably about between 5% to 15%, or even about between 10% to 20%, or even more preferably about between 15% to 25%, or still more preferably about between 20% to 30%, or even 35% or 40%, and as much as even about 50% or even more. In any of the above percent dosages, the papain and the bromelain can be formulated together in the same composition or formulated separately in different compositions.

In another embodiment, the papain and bromelain are formulated and/or administered in quantities based on their ratios. In one embodiment, the papain and bromelain of the present invention are formulated together in a single composition having a ratio of papain:bromelain of about 10:1, more preferably from about 9:1 to about 8:1, more preferably still from about 7:1 to about 6:1, still more preferably from about 5:1 to about 4:1 or 3:1, and even more preferably still from about 2:1 to about 1:1, and any suitable ratio therein between. In another embodiment, the papain and bromelain of the present invention are formulated together in a single composition having a ratio of papain:bromelain of about 1:10, more preferably from about 9:1 to about 8:1, more preferably still from about 7:1 to about 6:1, still more preferably from about 5:1 to about 4:1 or 3:1, and even more preferably still from about 2:1 to about 1:1, and any suitable ratio therein between.

In another embodiment, the papain and bromelain of the present invention are co-administered in a ratio of papain:bromelain of about 10:1, more preferably from about 9:1 to about 8:1, more preferably still from about 7:1 to about 6:1, still more preferably from about 5:1 to about 4:1 or 3:1, and even more preferably still from about 2:1 to about 1:1, and any suitable ratio therein between. In another embodiment, the papain and bromelain of the present invention are co-administered in a ratio of papain:bromelain of about 1:10, more preferably from about 9:1 to about 8:1, more preferably still from about 7:1 to about 6:1, still more preferably from about 5:1 to about 4:1 or 3:1, and even more preferably still from about 2:1 to about 1:1, and any suitable ratio therein between.

Methods of Use

The present invention further relates to methods of treating viral-induced lesions, for example, warts and molluscum lesions, by administering a therapeutically effective amount of papain and/or bromelain to a subject in need thereof. In some embodiments, the papain and/or bromelain are administered directly to a lesion, such as by applying a therapeutically effective amount of a cream or lotion or other dermatologically-acceptable formulation to the lesions. In another aspect, the papain and/or bromelain are delivered directly to the lesion, e.g. a wart or molluscum lesion, by injection of the active ingredients into the lesion. Direct delivery of the active ingredients also includes topical application of one or more of the active ingredients by transdermal delivery, e.g. by a patch, bandage, or other suitable transdermal system which brings the lesion into direct contact with the patch, bandage or other suitable transdermal system containing the active ingredients.

In one embodiment, the present invention is directed to a method of treating warts by administering a therapeutically effective amount of papain and bromelain to a subject in need thereof. In another embodiment, the present invention provides a method of treating molluscum lesions by administering a therapeutically effective amount of papain and bromelain to a subject in need thereof.

Preferably, the papain and bromelain are co-administered. As used herein, the term “co-administered” or “co-administration” is meant to refer to the administration of two or more agents at the same time or at substantially the same time, and in any order. “At the same time” encompasses situations where the papain and bromelain are administered at the same time as a single composition or mixture (e.g. a single cream, lotion or oil), as well as, separate compositions of papain and bromelain which are applied or administered at the same time (e.g. spotting onto the skin both a bromelain lotion and a papain oil and then rubbing them into the lesion at the same time). “At substantially the same time” can include situations where a first agent (e.g. papain) is administered to a subject in need thereof, followed by the administration of a second agent (e.g. bromelain) seconds, minutes, or hours or even a day or days later.

Where papain and bromelain are administered separately, they can be administered as the same type of formulation or different formulations. For example, the papain could be administered separately as a cream, whereas the bromelain could be separately administered as an oil, sunscreen, or by direct injection. Preferably, the papain and bromelain are directly administered to the lesions under treatment by the invention. By “directly administered” is meant directly applying a formulation of the papain and/or bromelain to the lesion of interest, e.g. directly to the wart or molluscum lesion, such that the papain and bromelain contact the lesion directly. Direct administration can include any topical application, direct injection, or transdermal patch system, or some other like route of administration.

The frequency of administering the papain and bromelain of the invention will be a function of various factors that can be easily determined and considered by the skilled artisan, such as, for example, the age of the patient (e.g. it may be preferable to administer the compositions of the invention to a child less frequently as compared to an adult), the type and severity of the lesion(s) (e.g. more severe lesions may preferably benefit from greater frequency of administration), the location of the lesion(s) on the body (e.g. more sensitive regions may preferably benefit from lower frequency of administration), etc. Moreover, the skilled artisan will be able to make a determination based on his or her knowledge and experience and good judgment as to the particular frequency of administration used on a case-by-case basis. In one embodiment, the papain and bromelain are administered at least once daily, more preferably at least twice daily, more preferably still at least three times daily to four times daily, even more preferably at least five to six times daily and even up to ten times daily. It will be appreciated by the skilled artisan that the frequency of administration may be limited by irritation or other undesirable side-effects that can develop more frequent exposure of the skin to the formulations of the invention.

Any suitable means for directly administering the papain and bromelain of the present invention (optionally together with any supplemental therapy as described below) is contemplated by the present invention. The methods of the invention, generally speaking, may be practiced using any mode of administration that is medically acceptable, meaning any mode that produces effective levels of the active compounds without causing clinically unacceptable adverse effects. Preferred means to directly administer the compositions of the invention include, but are not limited to, topical administration (e.g. creams, lotions or oils), injection into the lesion (e.g. by syringe), transdermal patches and/or bandages containing releasable quantities of the compositions of the invention.

In another aspect, the present invention relates to a method of co-administering the papain and bromelain compositions of the invention together with one or more supplemental therapies for treating the viral-induced lesions of the invention, especially warts and molluscum lesions. Such supplemental therapies can encompass any suitable strategy that is known or any future strategy equivalent to known therapies, including, for example, strategies that are aimed at reducing or eradicating the lesions by mechanical means (e.g. laser treatment, surgical excision) or chemical means (e.g. acid treatments), strategies that are aimed at inhibiting or eradicating the underlying viral infection (e.g. anti-viral compounds, small molecules, antibodies, or inhibitory nucleic acids), or strategies aimed at enhancing the immune system (e.g. interferons or imiquimod), or even combinations of one or more of the above strategies. One of ordinary skill in the art can determine the frequency and/or dosages of the one or more known therapies without undue experimentation.

The papain and bromelain can be administered separately or together, e.g. in a single composition or mixture. The particular order of administering the papain and bromelain of the invention together with a supplemental therapy is not important. For example, in one aspect, the papain and bromelain of the invention can be co-administered (e.g. each individually at about the same time, or each together as a single mixture or composition), together with the administering the one or more supplemental therapies at or about at the same time, before, or even after the papain/bromelain administration. If more than one supplemental therapy is used in combination with administering the papain and bromelain of the invention, the present invention contemplates administering a first supplemental therapy, followed by the papain and bromelain co-therapy, which can then be followed by a second supplemental therapy. Thus, the present invention encompasses any manner of timing and ordering in the co-administering of the papain and bromelain therapy of the invention together with one or more of the supplemental therapies.

In one aspect, the supplemental methods are mechanical methods for reducing or eradicating the lesions of the invention. Lesions of the invention, can be removed by mechanical means using surgical excision, tissue freezing methods (e.g. liquid nitrogen freeze via spray gun or cotton-tipped application at −196° C., cryogen with nitrous oxide tank freeze at −89° C., or aerosol spray with adapter freeze at −70° C.), burning methods (e.g. laser treatment, electrocautery, electrodesiccation), and cutting methods (e.g. curettage). Warts can be treated chemically, for example, by salicylic acid (e.g. COMPOUND W® or DUOFILM™ liquid or patches), bichloroacetic acid, trichloroacetic acid, tretinoin (Retin-A), dinitrochlorobenzene, cantharidin, podophyllin, 5-fluorouracil and bleomycin by way of topical or intralesional injections. Such treatments, especially acid treatments, require daily application for extended periods of time, ranging from weeks to many months, and further requires a continual removal of dead skin tissue. Newer treatment of warts involves methods of enhancing a patient's immune system, for example, by administering the topical agent imiquimod (Aldara), interferons (e.g. interferon alpha-2b or interferon alpha-N3), antigens (e.g. Candida antigen or mumps antigen), or cimetidine. Typically, immunotherapies are sought to suppress HPV re-infection once the gross lesions have been destroyed.

The supplemental methods can also include a method for inhibiting or eradicating the underlying viral infection. It will be appreciated that the lesions of the invention are caused by epidermis viral infections by viruses including HPV and MCV. Any anti-viral substance, including, for example, an antiviral compound, antiviral small-molecule drug, antiviral peptide, antiviral antibody or inhibitory fragment thereof, or an antiviral inhibitory nucleic acid that is capable of inhibiting or blocking the spread, replication or growth of HPV or MCV is contemplated by the present invention.

In one aspect, the supplemental methods involve administering an antiviral small-molecule drug or compound, e.g. a compound or small-molecule capable of inhibiting the spread, replication or growth of HPV or MCV. Any known small-molecule drugs or compounds could be used in the present invention. Examples of compounds and/or small-molecules directed against HPV that could be used as a supplemental therapy include those disclosed in U.S. Pat. No. 6,703,387,

In another aspect, the supplemental methods can utilize anti-viral peptides that are capable of inhibiting the growth, spread or replication of viruses causing the lesions of the invention, e.g. HPV or MCV, such as those found in U.S. Pat. No. 6,610,473 or 6,783,763, each of which are incorporated herein by reference. The supplemental methods can also utilize anti-viral antibodies or fragments thereof that are capable of inhibiting the growth, spread or replication of the viruses causing the lesions of the invention, e.g. HPV or MCV, such as those found in U.S. Pat. No. 6,531,127, which is incorporated herein by reference.

In another aspect, the supplemental methods can utilize anti-viral inhibitory nucleic acids that are capable of inhibiting the growth, spread or replication of the viruses causing the lesions of the invention, e.g. HPV or MCV, such as those found in U.S. Pat. Nos. 6,458,940, 5,364,758 and International Publication Nos. WO/91/08313, WO 93/20095, and WO 95/04748, each of which are incorporated by reference in their entireties. These agents can target any viral gene, and preferably those genes that are involved in viral replication and propagation such that interference thereof at the transcriptional or translational level will have a deleterious effect on the virus, e.g. the E1 or E2 proteins of HPV, each of which are required for viral replication (Mohr et al., 1990, Science 250:1694-1699, Seo et al., 1993, Proc. Natl. Acad. Sci., 90:2865-2869, 1995, J. Biol. Chem., 270(45):27283-27291, and Yasugi et al., 1997, J. Virol. 71, 891-899, each of which are incorporated here by reference). Antisense agents are thought to disrupt the function of a target by one of several mechanisms: by preventing the binding of factors required for normal transcription, splicing, or translation; by triggering the enzymatic destruction of mRNA by RNase H, or by destroying the target via reactive groups attached directly to the antisense oligonucleotide. Any type of antisense agent is contemplated, e.g. inhibitory oligonucleotides, synthetic or naturally isolated inhibitory agents, small inhibitory RNA (siRNA), or short-hairpin RNAs (shRNA), so long as it is capable of inhibiting the replication, growth or spread of a virus underlying the lesions of the invention, e.g. the viruses causing warts and MC lesions.

In embodiments relating to the treating of molluscum cantagiosum, any supplemental treatment that is typically or normally used to treat MC lesions can be used in combination with the methods of the present invention. MC is generally thought to be a self-limited disease, which, left untreated, will eventually resolve in immunocompetent hosts but may be protracted in atopic and immunocompromised individuals. Most of the known treatments of MC consist of various means to traumatize the lesions, but also can include antiviral and immune-modulating treatments options. The following is a brief summary of some of the more common treatments for MC that can be used together with the papain/bromelain therapy of the invention.

Cryotherapy

In one aspect, cryotherapy can be used together with the method of the invention. In one embodiment, liquid nitrogen, dry ice, or Frigiderm are applied to each individual lesion for a few seconds, prior to or after administering the papain/bromelain composition of the invention (Janniger C K, Schwartz R A. Molluscum Contagiosum in children. Cutis 1993; 52: 194-196). The skilled artisan will appreciate, however, that care must be given to avoid scarring.

Evisceration

The method of administering papain and/or bromelain of the invention can be carried out or co-administered with any standard method of physical removal of the lesions or a portion thereof. For example, the core can be eviscerated with an instrument such as a scalpel, sharp tooth pick, edge of a glass slide, or any other instrument capable of removing the umbilicated core. Because of its simplicity, patients, parents, and caregivers may be taught this method so new lesions can be treated at home (Valentine C L, Diven D G, Treatment modalities for molluscum contagiosum. Dermatologic Therapy 2000; 13: 285-289; Epstein W L. Molluscum contagiosum. Semin Dermatol 1992; 11: 184-189).

Curettage

Curettage is another method of removal that can be used together with the papain/bromelain compositions of the invention. It can be used with and without light electrodessication. This method is more painful, and it is recommended that a topical anesthetic cream be applied to the lesions before the procedure to decrease the pain. This method has the advantage of providing a reliable tissue sample to confirm the diagnosis (Valentine C L, Diven D G, Treatment modalities for molluscum contagiosum, Dermatologic Therapy 2000; 13: 285-289; Epstein W L. Molluscum contagiosum, Semin Dermatol 1992; 11: 184-189).

Podophyllin and Podofilox

In another embodiment, the method of administering the papain and/or bromelain compositions of the invention can be co-administered with phodophyllin and podofilox. For example, a 25% suspension of podophyllin in a tincture of benzoin or alcohol may be applied once a week. This treatment requires some precautions. It contains two mutagens, quercetin and kaempherol. Some of the listed side effects include severe erosive damage in adjacent normal skin that may cause scarring and systemic effects such as peripheral neuropathy, renal damage, adynamic illeus, leucopenia, and thrombocytopenia, especially if used generously on mucosal surfaces. Podofilox is a safer alternative to podophyllin and may be used by the patient at home. The recommended use usually consists of application of 0.05 ml of 5% podofilox in lactate buffered ethanol twice a day for 3 days (Valentine C L, Diven D G, Treatment modalities for molluscum contagiosum, Dermatologic Therapy 2000; 13: 285-289; Arndt K A. Manual of dermatologic therapeutics, 5th ed. Boston: Little Brown, 339-340, 1995).

Cantharidin

In yet another embodiment, the papain/bromelain compositions of the invention can be co-administered with cantharidin (0.9% solution of collodian and acetone), which is often used in treating MCV. This blister-inducing agent is applied carefully and sparingly to the dome of the lesion with or without occlusion and left in place for at least 4 hours before being washed off. Cantharidin can cause severe blistering. It should be tested on individual lesions before treating large numbers of lesions. It should not be used on the face. When tolerated, this treatment is repeated every week until the lesions clear (Silverburg N B, Sidbury R, Mancini A J. Childhood molluscum contagiosum: Experience with cantharidin therapy in 300 patients. J Am Acad Dermatol 2000; 43: 503-507).

Iodine Solution and Salicylic Acid Plaster

In still another embodiment, the papain/bromelain compositions of the invention can be co-administered with a low percent iodine solution (e.g. 10%), which is placed on the molluscum papules and, when dry, the site is covered with small pieces of salicylic acid plaster (e.g. 50%) and tape. The process is repeated daily after bathing. After the lesions have become erythematous in 3-7 days, only the iodine solution is applied. Resolution has been reported in a mean of 26 days (Ohkuma M. Molluscum contagiosum treated with iodine solution and salicylic acid plaster. Int J Dermatol 1990; 29:443-445). Care should be taken not to allow maceration and erosion as a result of the supplemental treatment.

Tretinoin

The present invention further contemplates co-administering the papain/bromelain of the invention together with tretinion (e.g. 0.1% or 0.05% cream), which has been used in the treatment of MCV. The tretinion can be applied twice daily to the lesions.

Cimetdine

The present invention also contemplates co-administering the papain/bromelain of the invention together with oral cimetidine, which has been used in extensive molluscum infections (Avella J, Binder H, Madsen J, Ashkenase P. Effect of histamine H2 receptor antagonists on delayed hypersensitivity. Lancet 1978:1:624-626). Because cimetadine interacts with many systemic medications, a review of the patient's other medications is recommended.

Potassium Hydroxide

Another supplemental treatment option that can be co-administered with the papain/bromelain of the invention is the use of potassium hydroxide. In one study, an aqueous solution of 10% KOH was applied topically twice daily to all lesions with a swab. The treatment was discontinued when an inflammatory response or superficial ulcer became evident. Resolution occurred in a mean of 30 days (Romiti R, Ribeiro A P, Grinblat B M. Treatment of molluscum contagiosum with potassium hydroxide: A clinical approach in 35 children. Pediatr Dermatol 1999; 16: 228-231).

Imiquimod

The present invention further contemplates any other supplemental therapy to be used in combination with the method of administering papain and bromelain in accordance with this invention, including, for example, imiquimod (Hengge et al., Self administered topical 5% imiquimod for the treatment of common warts and molluscum contagiosum, British Journal of Dermatology 2000; 143: 1026-1031; Tyring et al., A randomized, controlled, molecular study of condylomata acuminate clearance during treatment with imiquimod, J Infec Dis 1998; 178:551-5; and Barba et al., An open label safety study of topical imiquimod 5% cream in the treatment of Molluscum contagiosum in children, Dermatol Online J 2001; Vol 7(1), 20) and the nucleoside analog, cidofovir (Zabawski, A review of topical and intralesional cidofovir, Dermatology Online J 2000; 6(1):3; Zabawaski et al., Topical cidofovir for molluscum contagiosum in children, Pediatr Dermatol 1999; 16(5):414-415). Other agents affecting the immune system that can facilitate in the treatment of MC by papain and bromelain, include, for example, Candida antigen, mumps antigen, interferon alfa-2b, interferon alfa-N3, or cimetidine.

As with the papain and bromelain of the present invention, the supplemental therapies contemplated by the invention, e.g. the inhibitory nucleic acids and antiviral peptides, compounds, small molecules, and antibodies, can be delivered by any suitable means and in any suitable form. By suitable means, it is meant that the nucleic acids can be delivered alone or in combination with the papain and bromelain of the invention as a dermatologically-acceptable cream, ointment, salve, lotion, cerate, balm, sunscreen, oil, or pharmaceutically-acceptable solution. Alternatively, where small molecules or peptides are involved in the supplemental therapy, administration can by any typically-used pharmaceutical delivery route, e.g. parenterally, intramuscularly, by ingestion (pills, gels, tablets, etc.), by direct injection, or transdermally.

The present invention also contemplates methods that monitor the progress or efficacy of treatment. Any suitable means for monitoring the efficacy of treatment that would be known to the person of ordinary skill in the art can be employed. For example, the visible appearance of the lesions can be monitored over time to assess whether the treatment is working. For example, a doctor, nurse or other health practitioner, or even the subject being treated, can observe the physical characteristics of a lesion under treatment, e.g. a wart, to compare the lesion's size, color, shape, thickness, hardness, and patterns against the lesion's appearance prior to or at an earlier point during the treatment. Photographs can be taken to record the physical appearance of lesions over time and to allow easy comparisons to be made during treatment. Rapidity of treatment can be determined by assessing the physical appearance of the lesions with respect to time. Such analyses can be done using any of the treatments described herein, including treating with papain and bromelain alone or treating with papain and bromelain together in combination with a supplemental therapy, such as, immunotherapy, or the like.

The present invention also contemplates assessing or monitoring the effectiveness of the methods of the invention by determining or measuring the level of underlying viral infection. One of ordinary skill in the art will appreciate the various ways that the underlying virus can be tested, including, for example, screening lesion tissue samples by molecular or immunological techniques, including, for example, PCR amplification of viral genes or virus-specific nucleotide sequences, nucleic acid hybridization techniques, or screening for virus-specific polypeptides using antibody-specific screens, e.g. ELISA. Amplification methods are well known in the art and include techniques such as, for example, polymerase chain reaction (PCR) amplification and reverse transcription PCR (RT-PCR), as well as others. The amplified products may be detected and analyzed using any of the numerous techniques well known in the art. In one aspect, it may be of interest to the skilled artisan who is administering a combination treatment of the papain and bromelain of the invention together with an antiviral therapy, e.g. an antiviral antisense therapy against E1 or E2 protein, to monitor before, during or after treatment the level of HPV or MCV in the lesion. Methods for carrying out such detection schemes for detecting HPV or MCV in tissue samples are well-known in the art and can be found, for example, in U.S. Pat. No. 7,169,585 (“Papillomavirus vaccine”), U.S. Pat. No. 7,135,281 (“Screening for papilloma viruses”), U.S. Pat. No. 6,908,615 (“DNA encoding human papilloma virus type 18”), U.S. Pat. No. 6,830,887 (“Method and kit for quantitation and nucleic acid sequencing of nucleic acid analytes in a sample”), and U.S. Pat. No. 5,821,050 (“Drug screening assay for antiviral activity against papillomavirus”), each of which are incorporated herein by reference.

Lesions may be sampled for testing by any method known in the art, such as, for example, any well-known method for obtaining a biopsy. Numerous methods for obtaining a sample via biopsy are known in the art and include, for example, bite, brush, cone, cytological, aspiration, endoscopic, excisional, exploratory, incisional, percutaneous, punch, and surface biopsy. The skilled artisan will be knowledgeable as to the quantity of tissue required for analysis. Preparation of samples for the above analyses, e.g. immunological or molecular, is well known in the art, and any such technique may be used herein.

Kits of the Invention

In yet another embodiment, the present invention provides kits or packages containing the compositions of the invention and instructions for admixture and/or administration. Such kits contemplated by the invention can also contain any implement or device or tool for the successful and complete delivery of the compositions of the invention, such as, but not limited to, a syringe, sterile mixing vessel, measuring device, transdermal patch and instructions, etc. The kits of the invention are also not limited to the provision of a single dose or delivery of the compositions of the present invention, but can contain any suitable quantity of doses, such as, a suitable quantity of compositions to last 1 day to several days, 1 week, 1 month, or 1 year or more.

Any of the compositions of the kits of the invention can also include other suitable non-active ingredients and pharmaceutically acceptable carrier(s).

Further, the papain and/or bromelain can be provided in the same or different packages, and they can be provided pre-formulated (e.g. as a cream or lotion) or they can be provided as a dried form (e.g. powder), which can be mixed with a suitable pharmaceutically acceptable carrier prior to therapy.

While the description above refers to particular embodiments of the present invention, it will be understood that many modifications may be made without departing from the spirit thereof. The accompanying claims are intended to cover such modifications as would fall within the true scope and spirit of the present invention.

The present invention is additionally described by way of the following illustrative, non-limiting Examples that provide a better understanding of the present invention and of its many advantages.

Although the subject invention has been described with respect to preferred embodiments, those skilled in the art will readily appreciate that changes and modifications may be made thereto without departing from the spirit and scope of the subject invention as defined by the appended claims.

### EXAMPLES

### Example 1

### Use of Papain-Bromelain Composition to Treat Warts

Patient Group

The patients used in this example ranged in age from 3 years old to 50 years old. No patients were discriminated on the basis of age, severity of warts, number of warts, or whether or not the lesions were new or recurrent. With some patients, no prior wart therapies had been previously performed. For other patients, one or more prior treatments had been tried, however, such treatments were unsuccessful in removing the warts. The one or more prior treatments included over-the-counter topical salicylic acid preparations, cryotherapy, and laser treatment. Patients were given a choice to use a dermatogically-acceptable cream comprising a papain-bromelain composition having 30% papain (w/v) and 10% bromelain or any of the other treatment methods which were fully described to the patients. The patients did not know the exact ingredients of the administered composition.

The patients studied in this example had a variety of wart type including common warts, plantar warts, verruca plana (flat warts) and mosaic warts. A single patient often had multiple warts. Genital warts were excluded from this example. The warts treated were of various sizes, measuring from about 2 mm to more than about 2 cm in diameter. Mosaic warts treated in this example often covered an area of about 3-4 cm in size. The warts occurred on many different bodily sites including: hands and fingers; plantar—on the bottom surfaces of the feet; periungual areas; legs; face; arms and forearms.

Treatment

All patients were instructed to apply a papain-bromelain composition having papain and bromelain at concentrations of 30% and 10%, respectively, in a small amount directly to the warts with a cotton swab applicator twice a day. Patients were instructed not to “double dip” the cotton swab applicator into the composition. Patients were instructed to try to apply the cream only to the warts, minimizing cream in contact with normal skin. The patients were further instructed to apply a band-aid or piece of waterproof tape over the wart.

The first application of the composition was done in the presence of a doctor in order to demonstrate to the patient proper application technique. Plantar warts were pared down with a #15 surgical scalpel prior to the initial application of the compound.

Patients were advised to discontinue use of the compound if they developed irritation to the surrounding normal skin. Patients were asked to schedule a follow-up appointment to examine the treated sites in one month. If all warts had not resolved within one month, a second appointment was made one month later.

Clearance was described only if all warts had completely resolved. If some but not all lesions had resolved and a patient discontinued treatment, they were recorded as having no response. Decrease in size of warts without complete clearance was likewise recorded as no response.

Results

In total, 23% of the 53 patients were not compliant with treatment either because they did not use the papain/bromelain composition at the recommended frequency of application or did not continue for the full course of treatment.

Only 2 of the 53 treated patients discontinued use of the composition due to irritation, redness and mild burning, particularly of the skin surrounding the treated lesion(s).

Of the remaining compliant patients, 81% of the patients had complete clearance of all warts within 2 months of treatment using the composition in accordance with the present method. None of the lesions have recurred.

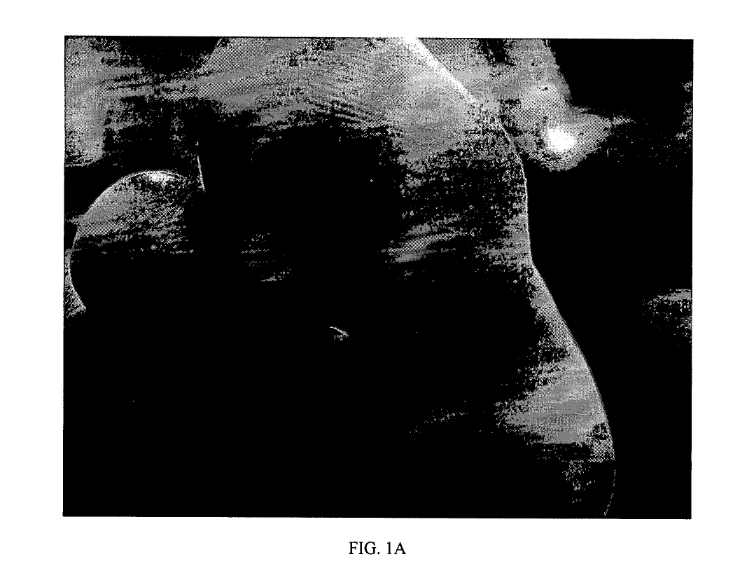

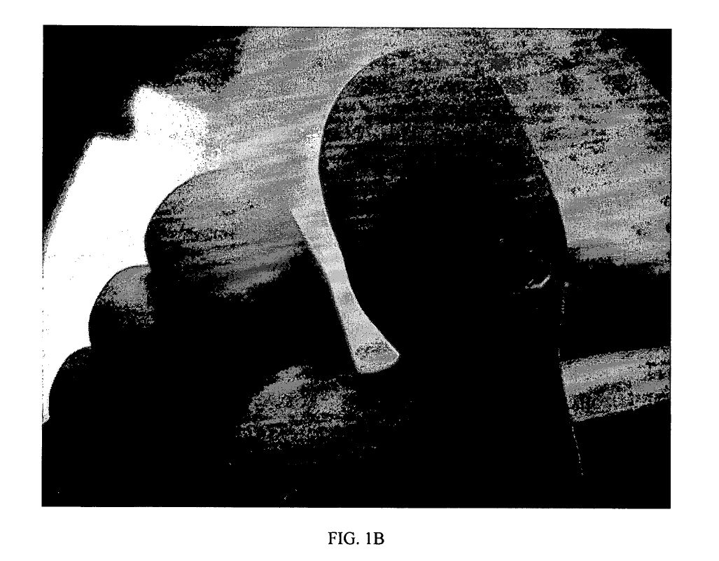

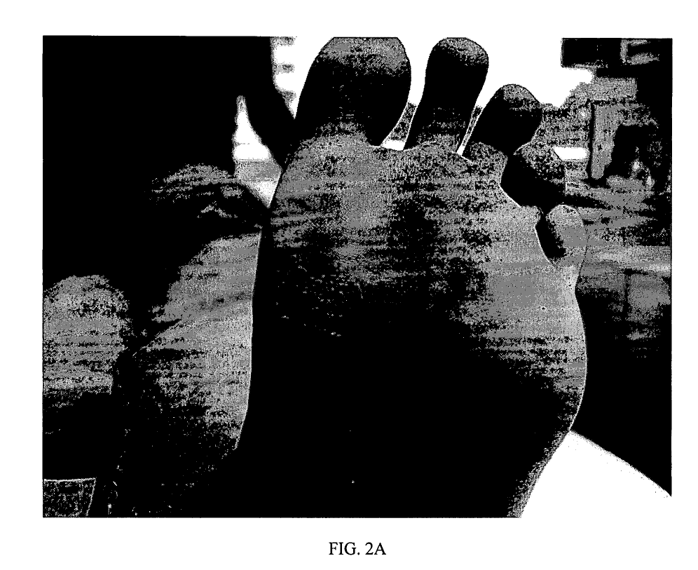

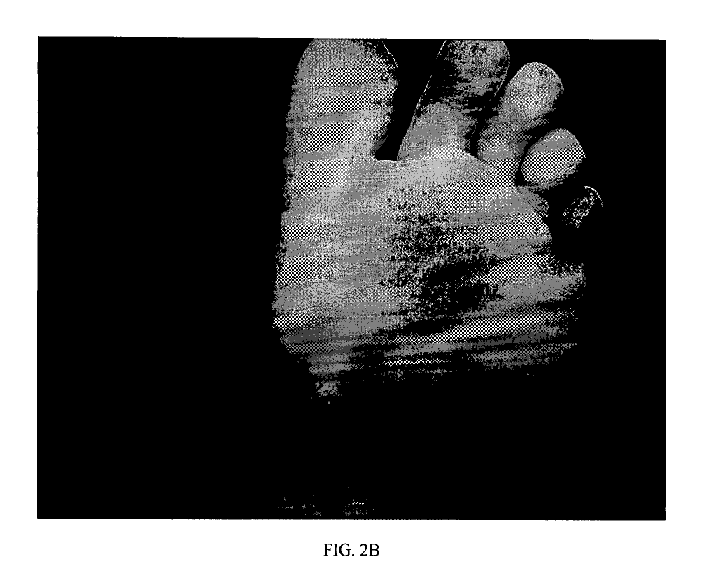

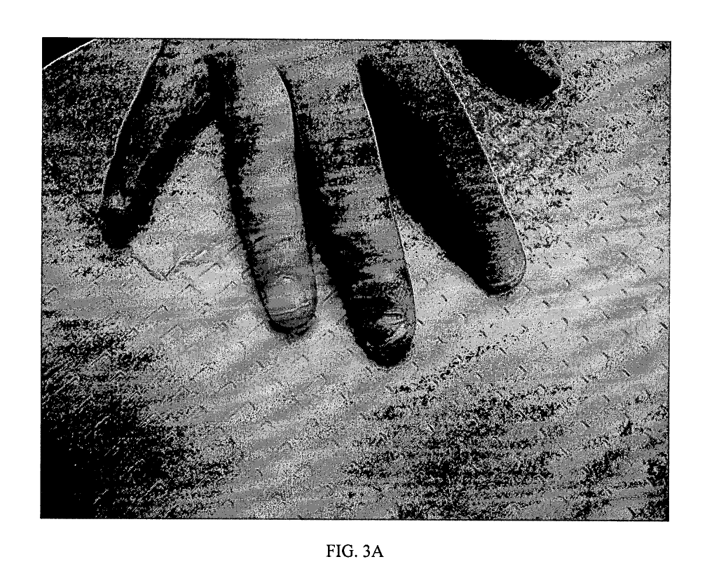

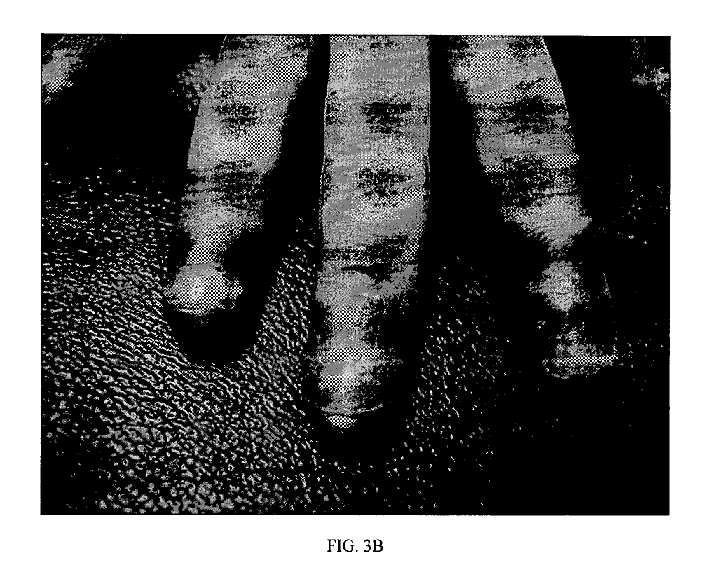

Photographs were taken of patients' lesions before and after treatment. See FIG. 1A, FIG. 2A, and FIG. 3A for photographs of three separate lesions on three different patients before treatment in accordance with this example. FIG. 1B, FIG. 2B, and FIG. 3B are photographs of the same lesions after treatment.

### Example 2

### Use of Papain-Bromelain Composition to Treat Molluscum Contagiosum

Patient Group

In this example, treated patients ranged in age from 2-12 years old. This age group is reflective of the typical ages of patients having molluscum contagiosum.

No patients were discriminated on the basis of severity of condition, number of lesions, or whether or not the lesions were new or recurrent. For most of the patients, this was the first line therapy, although some patients had failed topical Cantharidin or salicylic acid preparations. Patients were given an option to use the papain/bromelain composition or other treatment modalities including topical Cantharidin, salicylic acid, cryotherapy, and curettage. The ingredients in the compound were not disclosed to the patients.

Treatment

All molluscum lesions were treated with a papain/bromelain composition formulated as a cream and having papain and bromelain at concentrations of about 25% and 5%, respectively, of the total weight of the composition. Molluscum lesions were treated primarily on the trunk and extremities. Two patients having lesions on the face were also treated. No genital lesions were treated with the composition. All patients had multiple lesions.

The patients, and parents of young-aged children, were instructed to apply the compound in small amounts directly to the molluscum lesions using a cotton swab applicator once a day. Patients (and their parents) were instructed not to “double dip” the cotton swab applicator into the composition. Patients (and their parents) were instructed to try to apply the cream only to the molluscum lesions, and minimizing cream coming in contact with normal skin.

The first application was done in the presence of a doctor to demonstrate to the patient proper application technique. Patients were advised to discontinue use of the compound if they developed irritation to the surrounding normal skin. Patients were asked to schedule a follow-up appointment to examine the treated sites in one month. If all lesions had not resolved within one month, treatment application was increased to twice a day and a second appointment was made one month later.

Clearance was described only if all molluscum lesions had completely resolved. If some but not all lesions had resolved and a patient discontinued treatment, they were recorded as having no response. Decrease in size of lesions without complete clearance was likewise recorded as no response.

Results

In total, 8 patients were treated with the composition. All had multiple lesions. In total, 2 of the 8 treated patients discontinued use of the cream when applied twice a day due to irritation. Theses were the only patients who reported side-effects from the treatment. The irritation included redness and mild burning, particularly of the surrounding skin.

In total, 63% of the patients had complete clearance of all molluscum lesions.

Having thus described in detail preferred embodiments of the present invention, it is to be understood that the invention defined by the above paragraphs is not to be limited to particular details set forth in the above description as many apparent variations and equivalents thereof are possible without departing from the spirit or scope of the present invention.

We claim:

1. A composition for treating a subject having a viral-induced lesion resulting from a viral infection, said composition comprising a mixture of papain and bromelain and a pharmaceutically acceptable carrier.

2. The composition according to claim 1, wherein the viral-induced lesion is a wart.

3. The composition according to claim 2, wherein the wart is a non-genital wart.

4. The composition according to claim 3, wherein the non-genital wart is a common wart, a periungual wart, flat wart, filiform wart, or plantar wart.

5. The composition according to claim 2, wherein the wart is a genital wart.

6. The composition according to claim 1, wherein the viral-induced lesion is caused by a human papillomavirus infection of the epidermis.

7. The composition according to claim 1, wherein the viral-induced lesion is molluscum contagiosum.

8. The composition according to claim 1, wherein the subject is a human.

9. The composition according to claim 1, wherein the subject is a child.

10. The composition according to claim 1, wherein the subject is immunocompromised.

11. The composition according to claim 1, wherein the papain is at about between 25-35% (w/v) and the bromelain is at about between 5-15% (w/v) of the composition.

12. The composition according to claim 1, wherein the papain is at about 30% (w/v) and the bromelain is at about 10% (w/v) of the composition.

13. The composition according to claim 1, wherein the papain is at about between 20-30% (w/v) and the bromelain is at about between 5-10% (w/v) of the composition.

14. The composition according to claim 1, wherein the papain is at about 25% (w/v) and the bromelain is at about 5% (w/v) of the composition.

15. The composition according to claim 1, wherein the papain and the bromelain are present in a ratio of papain:bromelain of about 10:1, 9:1, 8:1, 7:1, 6:1, 5:1, 4:1, 3:1, 2:1, or 1:1.

16. The composition according to claim 1, wherein the pharmaceutically acceptable carrier is a lecithin organogel base.

17. The composition according to claim 1, wherein the composition is in the form of a dermatologically-acceptable cream, ointment, salve, lotion, cerate, balm, sunscreen, oil, or solution.

18. A method for treating a wart by administering to a subject in need thereof the composition of claim 1.

19. A method for treating molluscum contagiosum to a subject in need thereof the composition of claim 1.

20. A method for treating a viral-induced lesion resulting from a viral infection comprising administering papain and bromelain directly to the viral-induced lesion in a subject in need thereof.

21. The method according to claim 20, wherein the viral-induced lesion is a wart.

22. The method according to claim 21, wherein the wart is a non-genital wart.

23. The method according to claim 22, wherein the non-genital wart is a common wart, a periungual wart, flat wart, filiform wart, or plantar wart.

24. The method according to claim 21, wherein the wart is a genital wart.

25. The composition according to claim 20, wherein the viral-induced lesion is caused by a human papillomavirus infection of the epidermis.

26. The composition according to claim 20, wherein the viral-induced lesion is molluscum contagiosum.

27. The method according to claim 20, wherein the subject is a human.

28. The method according to claim 20, wherein the subject is a child.

29. The method according to claim 20, wherein the subject is immunocompromised.

30. The method according to claim 20, wherein the papain and bromelain are administered in a ratio of about 10:1, 9:1, 8:1, 7:1, 6:1, 5:1, 4:1, 3:1, 2:1, or 1:1.

31. The method according to claim 20, wherein the papain and bromelain are administered as a composition.

32. The method according to claim 31, wherein the composition comprises papain at about between 25-35% (w/v) and the bromelain at about between 5-15% (w/v) of the composition.

33. The method according to claim 31, wherein the composition comprises papain at about 30% (w/v) and the bromelain at about 10% (w/v) of the composition.

34. The method according to claim 31, wherein the composition comprises papain at about between 20-30% (w/v) and the bromelain at about between 5-10% (w/v) of the composition.

35. The method according to claim 31, wherein the composition comprises papain at about 25% (w/v) and the bromelain at about 5% (w/v) of the composition.

36. The method according to claim 31, wherein the composition further comprises a pharmaceutically acceptable carrier.

37. The method according to claim 36, wherein the pharmaceutically acceptable carrier is a lecithin organogel base.

38. The method according to claim 31, wherein the composition is in the form of a dermatologically-acceptable cream, ointment, salve, lotion, cerate, balm, sunscreen, oil, or solution.

39. The method according to claim 20, wherein the step of administering the papain and bromelain is by topical administration.

40. The method according to claim 20, wherein the step of administering the papain and bromelain is by injection.

41. The method according to claim 20, wherein the step of administering the papain and bromelain is by transdermal patch.

42. The method according to claim 20, further comprising the step of co-administering a secondary therapy.

43. The method according to claim 42, wherein the secondary therapy is mechanical eradication of the viral-induced lesion.

44. The method according to claim 43, wherein the mechanical eradication is by curettage, laser, cryotherapy, electrocautery, or electrodesiccation.

45. The method according to claim 43, wherein the secondary therapy is a chemical therapy.

46. The method according to claim 45, wherein the chemical therapy comprises administering trichloroacetic acid, bichloroacetic acid, tretinoin, or salicylic acid.

47. The method according to claim 42, wherein the secondary therapy is an immune therapy.

48. The method according to claim 47, wherein the immune therapy comprises administering imipuimod, Candida antigen, mumps antigen, interferon alfa-2b, interferon alfa-N3, or cimetidine.
